(19) United States
(12) Patent Application Publication
(10) Pub. No.: US 2011/0195409 A1
(43) Pub. Date: Aug. 11, 2011

(54) STREPTOCOCCUS PYOGENES CLASSIFICATION

(75) Inventors: Guido Grandi, Milano (IT); Marirosa Mora, Siena (IT); John L. Telford, Monteriggioni (IT); Chiara Zingaretti, Ravenna (IT); Fabiana Falugi, Monteriggioni (IT)

(73) Assignee: Novartis Vaccines and Diagnostics SRL, Siena (IT)

(21) Appl. No.: 12/741,514

(22) PCT Filed: Nov. 6, 2008
(86) PCT No.: PCT/GB2008/003769

§ 371 Date: Mar. 23, 2011

(30) Foreign Application Priority Data
Nov. 6, 2007 (GB) 0721757.6

(51) Int. Cl. C12Q 1/68; C07K 7/06; C07K 14/315; C07H 21/00; C07K 16/12; C09K 3/00

(52) U.S. Cl. 435/6.11; 530/329; 530/328; 530/327; 530/326; 530/325; 530/324; 530/350; 536/23.7; 530/387.9; 252/182.12

(57) ABSTRACT

The difference Lancefield T-serotypes correlate with the sequence of the pilus backbone protein (Pbp) in Streptococcus pyogenes (GAS). We have sequenced Pbp for over 50 GAS strains, representing the major disease-associated serotypes, and have identified 15 Pbp variants. These 15 variants have been shown to determine the specificity of the T-serotyping, such that sequencing of the Pbp from a given GAS strain reliably predicts that strain's T-serotype. thus the invention permits the t-serotype of a GAS strain to be determined based on genotype.

This application claims the benefit of United Kingdom patent application 0721757.3 filed on 6 Nov. 2007, the complete contents of which are incorporated herein by reference.

### TECHNICAL FIELD

This invention is in the field of classifying Streptococcus pyogenes (GAS) strains.

### BACKGROUND ART

More than 50 years ago Lancefield and colleagues classified GAS strains on the basis of serological recognition of the trypsin-sensitive M antigen and of a trypsin-resistant antigen known as the T antigen [1,2]. While the M-protein has been thoroughly studied over the last three decades, the basis of T-serotyping has not received the same attention.

There are about 20 known Lancefield T-serotypes (including 1, 2, 3, 4, 5, 6, 8, 9, 11, 12, 13, 14, 18, 22, 23, 25, 27, 28, 44, B₃₂₆₄ and Impetigo 19), some of which are overlapping or redundant. The T antigen has been used, in conjunction with the M antigen, to provide an additional tool for the sub-classification of GAS strains by an agglutination assay in which T-specific sera are used [3,4]. These T-typing sera are obtained after the streptococci are treated with trypsin, which digests the trypsin-sensitive protein molecules on the cell surface including the M protein, leaving the T antigen exposed. Furthermore, the T antigens form the basis of a major serological typing scheme that is used for those streptococci producing either no or a non-typeable M protein.

One problem with the T-serotyping system is that it relies on (i) the ability to maintain viable GAS organisms, in order to provide sufficient protein for analysis and (ii) good-quality, well-characterized antisera [5]. Moreover, some strains are often recognized by patterns of closely associated T sera rather than by single serum (e.g. T3/13/B3264, T5/27/44, T8/25/Imp19), and other strains may react non-specifically with many T sera leading to agglutination and, depending on the intensity of trypsinization, they may lose true T-protein reaction [6].

Reference 7 concludes that the Pbp, Pap1 and PrtF2 proteins all contribute to the T-type of GAS.

### DISCLOSURE OF THE INVENTION

The inventors have found that the different Lancefield T-serotypes correlate with the sequence of the pilus backbone protein (Pbp) in GAS. They have sequenced Pbp for over 50 GAS strains, representing the major disease associated serotypes, and have identified fifteen Pbp variants. Thirteen of these variants have been shown to determine the specificity of the T-serotyping, such that sequencing of the Pbp from a given GAS strain can predict that strain's T-serotype. Thus the invention permits the T-classification of a GAS strain to be determined based on genotype. Gene sequence analysis of the Pbp gene is much simpler than the existing serological assays.

The invention provides a method for determining the T-classification of a Streptococcus pyogenes bacterium, wherein the sequence of the bacterium's pbp gene is determined in whole or in part. The invention also provides a method for analysing a Streptococcus pyogenes bacterium, comprising a step in which the sequence of the bacterium's pbp gene is determined in whole or in part.

The invention also provides a method for determining the T-classification of a Streptococcus pyogenes bacterium, wherein the sequence, in whole or in part, of the bacterium's pbp gene is compared to one or more known pbp sequence(s). Usually it is compared to at least two known pbp sequences (e.g. at least 2, 3, 4, 5, 6, 7, 8, 9, 10, 11, 12, 13, 14, 15, 16, 17, 18, 19, 20 or more; preferably at least 5 sequences). If these known pbp sequences have been correlated with T-serotypes then the closest match between the bacterium's pbp sequence and the known pbp sequence permits the bacterium's T-serotype to be determined.

The invention also provides a kit for analysing a Streptococcus pyogenes bacterium, comprising primers for amplifying a nucleic acid sequence comprising the whole of part of a pbp gene from a Streptococcus pyogenes bacterium.

The sequence of the pbp gene can be compared to known sequences and the T-classification can thereby be determined. As described in more detail below, enough of the gene must be sequenced to permit it to be distinguished from the pbp genes of other T-serotypes.

The invention also provides a method for determining if a Streptococcus pyogenes bacterium has a particular T-serotype, comprising a step in which the sequence of the bacterium's pbp gene is compared to the sequence of a pbp gene from a Streptococcus pyogenes strain that is known to have the particular T-serotype, with a sequence match indicating that the bacterium has the particular T-serotype and no sequence match indicating that the bacterium does not have the particular T-serotype. Further details are provided below.

The invention also provides a method for determining if a Streptococcus pyogenes bacterium has a particular T-serotype, comprising a step in which the bacterium's pbp gene is contacted with a nucleic acid that, under the conditions of this step, gives a first signal when contacted with one of SEQ ID NOs: 58 to 70 and a second signal when contacted with each of the other SEQ ID NOs: 58 to 70, wherein the first and second signals can be distinguished from each other. Thus, for example, the step may use a primer or probe that is specific to only one of SEQ ID NOs: 58 to 70, with amplification or hybridisation being the first signal and lack of amplification or hybridisation being the second signal. This method may be performed directly on S. pyogenes nucleic acid, but will usually be performed on nucleic acid amplified therefrom. The second signal may include a variety of different sub-signals, but each of these can be distinguished from the first signal.

A relationship between sequence and T-serotype has previously been suggested. Schneewind et al. [8] cloned a gene coding for a protein recognized by the T6 antisera and located the gene in the FCT (Fibronectin-binding, Collagen-binding T-antigen) region of a M6 strain. Reference 9 reports that the T6 antigen is Pbp and that three variants of this protein are specifically recognized by three other T antisera. However, the extent of Pbp variability remained unclear until the present work, and nor was the relationship of Pbp variation to Lancefield T-serotypes fully understood. Even so, in some embodiments, the invention does not relate to the analysis of a strain with one of the six following T-serotypes: 1; 5; 6; 12; 27; 44. T-serotypes 5, 27 and 44 are closely related.

### GAS Tee-Types

The inventors have sequenced the pbp gene from over 50 GAS strains and have identified 15 distinct variants. The prototype amino acid sequences for each of the variants are SEQ ID NOs 1 to 15, encoded by SEQ ID NOs 58 to 72, respectively. 13 of the 15 variants have been correlated with T-serotypes as follows:

| | SEQ ID | | | | | | | | | | | | |
|---|---|---|---|---|---|---|---|---|---|---|---|---|---|
| | 1 | 2 | 3 | 4 | 5 | 6 | 7 | 8 | 9 | 10 | 11 | 12 | 13 |
| T-type | 1 | 2 | 3^{a} | 4 | 5^{b} | 6 | 8^{c} | 9 | 11 | 12 | 14 | 23 | 28 |

^{a}This variant also seen in T-serotype 13
^{b}This variant also seen in T-serotypes 27 and 44
^{c}This variant also seen in T-serotype 25

Sequence identity of at least 90% at the amino acid level has been observed within each of these 13 variants, but between the different variants it is generally less than 72% (see Table II).

Because of the intra-variant conservation and inter-variant variation, the sequence of a bacterium's pbp gene can readily be placed into one of these 13 groups, and thereby its T-classification can be assessed. In the event that a particular strain does not fit into any of the 13 groups then, in a similar way to the current T-system, it can be classified, at least preliminarily, as being either in one of the T-types not shown above or as being non-typeable.

A pbp gene may be sequenced in whole or in part. If partial sequence is used then its size and location within the pbp gene must be sufficient to place it in one (or none) of the 13 variants. The alignments in FIG. 4 shows the regions of pbp that are conserved between variants (i.e. unsuitable for distinguishing between them) and those that vary (i.e. can be used to distinguish the 13 variants). FIG. 4 aligns the variants within FCT types, because sequences in different FCT types align poorly and thus can readily be distinguished, whereas sequences in the same FCT type show several regions of overlap. For examples the first 180 and last 125 residues of the sequences encoding SEQ ID NOs: 3, 5, 8, 9, 10, 11, 13 and 14 are very conserved (FIG. 4A) and so a typical method to distinguish these sequences will focus on different regions. The coding sequences for SEQ ID NOs: 1 and 4 are sufficiently different from each other and from the other SEQ ID NOs: that they can readily be distinguished.

In some cases more than one partial sequence may be determined, such that the combination of the two partial sequences is enough to determine the sequence's variant type, even though each individual partial sequence might, on its own, not be enough.

Where the invention refers to comparing the sequence of two nucleic acid sequences, this comparison may be performed at a nucleic acid level, or may be performed after transforming the sequence e.g. by comparing inferred amino acid sequences encoded by the two nucleic acids, or by comparing complements and reverse complements, etc.

### Nucleic Acid Detection

Various methods are known in the art for the sequence-specific detection of nucleic acids. Any of these can be used with the invention.

One of the advantages of using nucleic acid for identifying the T-serotype of a strain rather than immunological techniques is that, because efficient nucleic acid amplification techniques are widely available, viable GAS organisms do not have to be maintained in order to provide sufficient material for analysis. On the contrary, nucleic acids can be amplified and detected even with very low amounts of original GAS material.

Thus a method of the invention may involve a step of amplifying nucleic acid present in a sample. Suitable techniques include PCR, SDA, SSSR, LCR, TMA, NASBA, T7 amplification, etc. The technique preferably gives exponential amplification. The technique may be quantitative and/or real-time. Kits and methods for amplification and detection of bacterial sequences are known in the art e.g. it is known to characterise GAS strains by emm-specific PCR [10]. Array-based techniques can also be used.

Amplification techniques generally involve the use of at least one primer. With two primers and a double-stranded target, the primers hybridize to different strands of the target and are then extended. The extended products then serve as targets for further rounds of hybridization/extension, permitting exponential amplification. The net effect is to produce an amplicon from the target, the 5′ and 3′ termini of the amplicon being defined by the locations of the two primers in the target.

Thus the invention provides a kit comprising primers for amplifying a template sequence comprising at least a part of the S. pyogenes pbp gene, the kit comprising a first primer and a second primer, wherein the first primer comprises a sequence substantially complementary to a portion of said template sequence and the second primer comprises a sequence substantially complementary to a portion of the complement of said template sequence, wherein the sequences within said primers which have substantial complementarity define the termini of the template sequence to be amplified.

Kits and methods may use primers that are specific to one pbp variant, meaning that amplification will occur when that variant is present in a sample but will not occur if the variant is absent (e.g. if a different pbp variant is present). In other embodiments, they may use primers that are not specific to any particular pbp variant, meaning that amplification will occur when various pbp variants are present in a sample. Where such non-variant-specific primers are used then the variants can be distinguished from each other by characterising the amplicons e.g. by means of variant-specific probes, by sequencing the amplicons, etc. Examples of variant-specific primers are given below.

Primers for amplifying sequences from the pbp gene may be located inside the gene or outside the gene, provided that their amplicon comprises the whole or part of the pbp gene. FIG. 1 shows the genetic environment of the pbp gene in different FCT types, and primers outside the pbp gene may be located accordingly.

Kits of the invention may further comprise primers and/or probes for generating and detecting an internal standard, in order to aid quantitative measurements.

Kits of the invention may further comprise a probe which is substantially complementary to the template sequence and/or to its complement and which can hybridize thereto. This probe can be used in a hybridization technique to detect an amplicon. Such a probe may be variant-specific.

Kits of the invention may comprise more than one pair of primers (multiplex). Multiple pairs can be used for nested amplification of a target sequence, or can be used to amplify different target sequences. For instance, a kit or method may use a plurality of primer pairs, each pair permitting amplification of different pbp variants, thereby ensuring that a single set of reagents can amplify a range of different pbp variants. Where a plurality of primer pairs is used, it is possible to have a common primer in two pairs, but at least one primer will differ in each pair, thereby giving different amplicons.

Kits of the invention may also include one or more reagents for determining a strain's M-type. Kits and reagents for emm-typing are commercially available.

Because of the nature of nucleic acid hybridisation, if a primer(s) or probe is used that is specific to a particular pbp gene, a positive signal (e.g. generation of an amplicon, or hybridisation to a probe) means that the sequence of that particular gene has been determined in whole or in part, without actual base-by-base sequencing. Such sequence-specific reagents thus provide indirect sequence determination as a result of the sequence-specific nature of their behaviour.

Example primers include SEQ ID NO:s 115-160, as shown in Table I.

### Tee-Type Detection

The invention provides a method for determining if a test GAS has a particular T-serotype by comparing the sequence of its pbp gene to the sequence of a pbp gene from a GAS that has a known T-serotype. If the sequence from the test GAS matches the known sequence then they have the same T-serotype; if the sequences do not match then they have different T-serotypes.

The invention thus provides a method for determining if a Streptococcus pyogenes bacterium has T-serotype 1, comprising a step in which the sequence of the bacterium's pbp gene is compared to the sequence of a pbp gene from a S. pyogenes strain in T-serotype 1 (e.g. to SEQ ID NO: 58), with a match indicating that the bacterium has T-serotype 1 and no match indicating that the bacterium does not have T-serotype 1.

The invention also provides a method for determining if a Streptococcus pyogenes bacterium has T-serotype 2, comprising a step in which the sequence of the bacterium's pbp gene is compared to the sequence of a pbp gene from a S. pyogenes strain in T-serotype 2 (e.g. to SEQ ID NO: 59), with a match indicating that the bacterium has T-serotype 2 and no match indicating that the bacterium does not have T-serotype 2.

The invention also provides a method for determining if a Streptococcus pyogenes bacterium has T-serotype 3 or 13, comprising a step in which the sequence of the bacterium's pbp gene is compared to the sequence of a pbp gene from a S. pyogenes strain in T-serotype 3 or 13 (e.g. to SEQ ID NO: 60), with a match indicating that the bacterium has T-serotype 3 or 13 and no match indicating that the bacterium does not have T-serotype 3 or 13.

The invention also provides a method for determining if a Streptococcus pyogenes bacterium has T-serotype 4, comprising a step in which the sequence of the bacterium's pbp gene is compared to the sequence of a pbp gene from a S. pyogenes strain in T-serotype 4 (e.g. to SEQ ID NO: 61), with a match indicating that the bacterium has T-serotype 4 and no match indicating that the bacterium does not have T-serotype 4.

The invention also provides a method for determining if a Streptococcus pyogenes bacterium has T-serotype 5, 27 or 44, comprising a step in which the sequence of the bacterium's pbp gene is compared to the sequence of a pbp gene from a S. pyogenes strain in T-serotype 5, 27 or 44 (e.g. to SEQ ID NO: 62), with a match indicating that the bacterium has T-serotype 5, 27 or 44 and no match indicating that the bacterium does not have T-serotype 5, 27 or 44.

The invention also provides a method for determining if a Streptococcus pyogenes bacterium has T-serotype 6, comprising a step in which the sequence of the bacterium's pbp gene is compared to the sequence of a pbp gene from a S. pyogenes strain in T-serotype 6 (e.g. to SEQ ID NO: 63), with a match indicating that the bacterium has T-serotype 6 and no match indicating that the bacterium does not have T-serotype 6.

The invention also provides a method for determining if a Streptococcus pyogenes bacterium has T-serotype 8 or 25, comprising a step in which the sequence of the bacterium's pbp gene is compared to the sequence of a pbp gene from a S. pyogenes strain in T-serotype 8 or 25 (e.g. to SEQ ID NO: 64), with a match indicating that the bacterium has T-serotype 8 or 25 and no match indicating that the bacterium does not have T-serotype 8 or 25.

The invention also provides a method for determining if a Streptococcus pyogenes bacterium has T-serotype 9, comprising a step in which the sequence of the bacterium's pbp gene is compared to the sequence of a pbp gene from a S. pyogenes strain in T-serotype 9 (e.g. to SEQ ID NO: 65), with a match indicating that the bacterium has T-serotype 9 and no match indicating that the bacterium does not have T-serotype 9.

The invention also provides a method for determining if a Streptococcus pyogenes bacterium has T-serotype 11, comprising a step in which the sequence of the bacterium's pbp gene is compared to the sequence of a pbp gene from a S. pyogenes strain in T-serotype 11 (e.g. to SEQ ID NO: 66), with a match indicating that the bacterium has T-serotype 11 and no match indicating that the bacterium does not have T-serotype 11.

The invention also provides a method for determining if a Streptococcus pyogenes bacterium has T-serotype 12, comprising a step in which the sequence of the bacterium's pbp gene is compared to the sequence of a pbp gene from a S. pyogenes strain in T-serotype 12 (e.g. to SEQ ID NO: 67), with a match indicating that the bacterium has T-serotype 12 and no match indicating that the bacterium does not have T-serotype 12.

The invention also provides a method for determining if a Streptococcus pyogenes bacterium has T-serotype 14, comprising a step in which the sequence of the bacterium's pbp gene is compared to the sequence of a pbp gene from a S. pyogenes strain in T-serotype 14 (e.g. to SEQ ID NO: 68), with a match indicating that the bacterium has T-serotype 14 and no match indicating that the bacterium does not have T-serotype 14.

The invention also provides a method for determining if a Streptococcus pyogenes bacterium has T-serotype 23, comprising a step in which the sequence of the bacterium's pbp gene is compared to the sequence of a pbp gene from a S. pyogenes strain in T-serotype 23 (e.g. to SEQ ID NO: 69), with a match indicating that the bacterium has T-serotype 23 and no match indicating that the bacterium does not have T-serotype 23.

The invention also provides a method for determining if a Streptococcus pyogenes bacterium has T-serotype 3, 5, 13 or 28, comprising a step in which the sequence of the bacterium's pbp gene is compared to the sequence of a pbp gene from a S. pyogenes strain in T-serotype 3, 5, 13 or 28 (e.g. to SEQ ID NO: 70), with a match indicating that the bacterium has T-serotype 3, 5, 13 or 28 and no match indicating that the bacterium does not have T-serotype 3, 5, 13 or 28.

Where the test GAS's pbp sequence is compared to the known pbp sequence, this comparison may be against one of the known pbp sequences disclosed herein (e.g. against a sequence encoding one of SEQ ID NOs: 1 to 13), or it may be against a different known pbp sequence that has sequence identity to one of the SEQ ID NOs: 1 to 13 coding sequences. Because of the low level of inter-variant sequence identity, the comparison sequence can differ substantially from SEQ ID NOs: 1 to 13 while still providing a useful result. For instance, SEQ ID NO: 1 has ≦40% identity to the other 12 sequenced pbp variants and so the comparison sequence for T-serotype I can code for SEQ ID NO: 1 or for a sequence having at least 70% identity to SEQ ID NO: 1 (e.g. at least 75%, 80%, 85%, 90%, 95%, 96%, 97%, 98%, 99% or more). The following table shows this information for full-length SEQ ID NO: 1 and for the other SEQ ID NOs 2 to 13:

| SEQ ID | Highest match | Threshold | e.g. at least % |
|---|---|---|---|
| 1 | 40% | 70% | 75%, 80%, 85%, 90%, 95%, 96%, 97%, 98%, 99% |
| 2 | 40% | 70% | 75%, 80%, 85%, 90%, 95%, 96%, 97%, 98%, 99% |
| 3 | 72% | 86% | 88%, 90%, 95%, 96%, 97%, 98%, 99% |
| 4 | 26% | 63% | 65%, 70% 75%, 80%, 85%, 90%, 95%, 96%, 97%, 98%, 99% |
| 5 | 69% | 85% | 87%, 88%, 89%, 90%, 95%, 96%, 97%, 98%, 99% |
| 6 | 55% | 88% | 90%, 95%, 96%, 97%, 98%, 99% |
| 7 | 40% | 70% | 75%, 80%, 85%, 90%, 95%, 96%, 97%, 98%, 99% |
| 8 | 60% | 80% | 85%, 90%, 95%, 96%, 97%, 98%, 99% |
| 9 | 57% | 79% | 80%, 85%, 90%, 95%, 96%, 97%, 98%, 99% |
| 10 | 65% | 83% | 85%, 90%, 95%, 96%, 97%, 98%, 99% |
| 11 | 67% | 84% | 85%, 90%, 95%, 96%, 97%, 98%, 99% |
| 12 | 55% | 88% | 90%, 95%, 96%, 97%, 98%, 99% |
| 13 | 72% | 86% | 88%, 90%, 95%, 96%, 97%, 98%, 99% |

This table also shows how a sequence match may be defined. If a comparison is being made against a pbp gene known to have T-type 1 (e.g. SEQ ID NO: 1, encoded by SEQ ID NO: 58) then a sequence identity of at least 70% can be considered as a match. In contrast, If a comparison is being made against a pbp gene known to have T-type 28 (e.g. SEQ ID NO: 13, encoded by SEQ ID NO: 70) then a sequence identity of at least 70% would not be a match.

If comparison is being made indirectly, by determining if a hybridisation event takes place (rather than by direct comparison of sequence information), these figures for inter-variant sequence identity can be used as the basis of stringency conditions for the hybridisation. For instance, SEQ ID NO: 58 (T-type 1) has no more than 40% identity to the pbp gene from any other T-type at an amino acid level, and so stringency conditions can be selected that will permit a primer or probe to hybridise to a target even when there are substantial differences. In contrast, higher stringency should be used with SEQ ID NO: 70 so as to avoid hybridisation with the pbp sequences from other T-types.

### Type-Specific Nucleic Acid Reagents

The invention provides a method for determining if test GAS bacterium has a particular T-serotype in which its pbp gene (including amplicons of the whole or part thereof) is contacted with a type-specific nucleic acid reagent i.e. a reagent that gives a particular signal when if encounters a nucleic acid target of a desired pbp variant but gives a different signal (e.g. no signal) when if encounters a nucleic acid target of a different pbp variant.

For instance, if the reagent were contacted (under the same hybridisation conditions) with each of SEQ ID NOs: 1 to 13 then it would give a particular signal for one of these thirteen target sequences but would give a different signal for the other thirteen. Thus the presence of this signal will indicate that the relevant target is present.

For example, the reagent may be a probe that can hybridise to only one of SEQ ID NOs: 58 to 70. The reagent may be a primer that can hybridise to only one of SEQ ID NOs: 58 to 70, or that has a 3′ sequence that permits extension only when it is hybridised to a particular one of SEQ ID NOs: 58 to 70. Thus the reagent may be used in combination with one or more further reagent(s) e.g. with a second primer.

Examples of variant-specific primers are indicated in FIG. 4. These can be used in their indicated orientation or in reverse complement orientation, as required.

FIG. 4A shows a forwards primer (SEQ ID NO: 161) that is common to the coding sequences of SEQ ID NOs: 3, 5, 8, 9, 10, 11, 13 and 14. This is used in combination with seven different reverse primers, as follows:

| | Target | | | | | | |
|---|---|---|---|---|---|---|---|
| | 3 | 5 | 8 | 9 | 10 | 11/14 | 13 |
| Rev primer SEQ ID: | 166 | 165 | 168 | 162 | 163 | 167 | 164 |
| Amplicon length | 685 | 530 | 865 | 145 | 308 | 767 | 395 |

Where the target sequence is in the same variant as SEQ ID NO: 11 or 14 then the primers will amplify in the same way. A probe specific to the region around nucleotides 700 of these two SEQ ID NOs can then be used to distinguish them.

FIG. 4B shows a reverse primer (SEQ ID NO: 169) that is common to the coding sequences of SEQ ID NOs: 2 and 7. This is used in combination with two different forward primers, as follows:

| Target | 2 | 7 |
|---|---|---|
| Fwd primer SEQ ID: | 170 | 171 |
| Amplicon length | 1674 | 1465 |

FIG. 4C shows a forwards primer (SEQ ID NO: 172) that is common to the coding sequences of SEQ ID NOs: 6 and 12. This is used in combination with two different reverse primers, as follows:

| Target | 6 | 12 |
|---|---|---|
| Rev primer SEQ ID: | 173 | 174 |
| Amplicon length | 1144 | 1245 |

Primer pair SEQ ID NOs 175 & 176 can be used to amplify the coding sequence of SEQ ID NO: 5.

Primer pair SEQ ID NOs 177 & 178 can be used to amplify the coding sequence of SEQ ID NO: 2.

The invention also provides a nucleic acid probe that can hybridise to only one of SEQ ID NOs: 58 to 70. The invention also provides a nucleic acid amplification primer that can hybridise to only one of SEQ ID NOs: 58 to 70. The invention also provides a nucleic acid amplification primer that has a 3′ sequence that permits extension when it is hybridised to only one of SEQ ID NOs: 58 to 70. The invention also provides a nucleic acid amplification primer that has a 3′ or 5′ sequence that permits ligation when it is hybridised to only one of SEQ ID NOs: 58 to 70. These variant-specific probes and primers thus permit the 13 different pbp variants to be uniquely identified.

### Polypeptides

The invention provides a polypeptide comprising an amino acid sequence having at least a % sequence identity to an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, and 252. For each of these SEQ ID NOs, the value of a may be independently selected from 50, 60, 70, 75, 80, 85, 90, 95, 96, 97, 98, 99, 99.5 or even 100. Within this list of SEQ ID NOs, numbers 1-57 are Pbp sequences, 179-216 are Pap1 sequences, and 217-252 are Pap2 sequences.

The invention also provides a polypeptide comprising a fragment of at least b consecutive amino acids of an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, and 252. For each of these SEQ ID NOs, the value of b may be independently selected from 5, 6, 7, 8, 9, 10, 12, 14, 16, 18, 20, 22, 24, 26, 28, 30, 35, 40, 45, 50, 60, 70, 80, 90, 100, 110, 120, 130, 140, 150 or more. The fragment, may comprise at least one T-cell or, preferably, a B-cell epitope of the sequence. T- and B-cell epitopes can be identified empirically (e.g. using PEPSCAN [11,12] or similar methods), or they can be predicted (e.g. using the Jameson-Wolf antigenic index [13], matrix-based approaches [14], TEPITOPE [15], neural networks [16], OptiMer & EpiMer [17,18], ADEPT [19], Tsites [20], hydrophilicity [21], antigenic index [22] or the methods disclosed in reference 23, etc.).

A polypeptide of the invention may meet both the sequence identity criterion and the fragment length criterion e.g. the invention also provides a polypeptide comprising an amino acid sequence having at least a % sequence identity to a particular SEQ ID NO: and comprising a fragment of at least b consecutive amino acids from that SEQ ID NO.

These polypeptides include homologs, orthologs, allelic variants and mutants. Typically, 50% identity or more between two polypeptide sequences is considered to be an indication of functional equivalence. Identity between polypeptides is preferably determined by the Smith-Waterman homology search algorithm as implemented in the MPSRCH program (Oxford Molecular), using an affine gap search with parameters gap open penalty=12 and gap extension penalty=1.

Polypeptides of the invention may, compared to SEQ ID NOs: 1-57 or 179-252, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may have one or more (e.g. 1, 2, 3, 4, 5, 6, 7, 8, 9, 10, etc.) single amino acid deletions relative to a reference sequence. The polypeptides may also include one or more (e.g. 1, 2, 3, 4, 5, 6, 7, 8, 9, 10, etc.) insertions (e.g. each of 1, 2, 3, 4 or 5 amino acids) relative to a reference sequence.

Polypeptides of the invention can be prepared in many ways e.g. by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. A preferred method for production of peptides <40 amino acids long involves in vitro chemical synthesis [24,25]. Solid-phase peptide synthesis is particularly preferred, such as methods based on tBoc or Fmoc [26] chemistry. Enzymatic synthesis [27] may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) [28]. Where D-amino acids are included, however, it is preferred to use chemical synthesis. Polypeptides of the invention may have covalent modifications at the C-terminus and/or N-terminus.

Polypeptides of the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides of the invention are preferably provided in purified or substantially purified form i.e. substantially free from other polypeptides (e.g. free from naturally-occurring polypeptides), particularly from other GAS or host cell polypeptides, and are generally at least about 50% pure (by weight), and usually at least about 90% pure i.e. less than about 50%, and more preferably less than about 10% (e.g. 5% or less) of a composition is made up of other expressed polypeptides. Polypeptides of the invention are preferably GAS polypeptides.

Polypeptides of the invention may be attached to a solid support. Polypeptides of the invention may comprise a detectable label (e.g. a radioactive or fluorescent label, or a biotin label).

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains. Polypeptides of the invention can be naturally or non-naturally glycosylated (i.e. the polypeptide has a glycosylation pattern that differs from the glycosylation pattern found in the corresponding naturally occurring polypeptide).

Polypeptides of the invention may be at least 40 amino acids long (e.g. at least 40, 50, 60, 70, 80, 90, 100, 120, 140, 160, 180, 200, 220, 240, 260, 280, 300, 350, 400, 450, 500 or more). Polypeptides of the invention may be shorter than 500 amino acids (e.g. no longer than 40, 50, 60, 70, 80, 90, 100, 120, 140, 160, 180, 200, 220, 240, 260, 280, 300, 350, 400 or 450 amino acids).

The invention provides polypeptides comprising a sequence —X—Y— of —Y—X—, wherein: —X— is an amino acid sequence as defined above and —Y— is not a sequence as defined above i.e. the invention provides fusion proteins. Where the N-terminus codon of a polypeptide-coding sequence is not ATG then that codon will be translated as the standard amino acid for that codon rather than as a Met, which occurs when the codon is translated as a start codon.

The invention provides a process for producing polypeptides of the invention, comprising culturing a host cell of to the invention under conditions which induce polypeptide expression.

The invention provides a process for producing a polypeptide of the invention, wherein the polypeptide is synthesised in part or in whole using chemical means.

The invention provides a composition comprising two or more polypeptides of the invention.

### Nucleic Acids

The invention also provides a nucleic acid comprising a nucleotide sequence encoding the polypeptides of the invention e.g. SEQ ID NOs: 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325 and 326. The invention also provides nucleic acid comprising nucleotide sequences having sequence identity to such nucleotide sequences. Such nucleic acids include those using alternative codons to encode the same amino acid.

The invention also provides nucleic acid which can hybridize to these nucleic acids. Hybridization reactions can be performed under conditions of different “stringency”. Conditions that increase stringency of a hybridization reaction of widely known and published in the art. Examples of relevant conditions include (in order of increasing stringency): incubation temperatures of 25° C., 37° C., 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 0.1×SSC, 0.1×SSC, or de-ionized water. Hybridization techniques and their optimization are well known in the art [e.g. see refs 29 & 30, etc.].

Nucleic acid comprising fragments of these sequences are also provided. These should comprise at least n consecutive nucleotides from the sequences and, depending on the particular sequence, n is 10 or more (e.g. 12, 14, 15, 18, 20, 25, 30, 35, 40, 50, 60, 70, 80, 90, 100, 150, 200 or more).

The invention provides nucleic acid of formula 5′-X—Y—Z-3′, wherein: —X— is a nucleotide sequence consisting of x nucleotides; —Z— is a nucleotide sequence consisting of z nucleotides; —Y— is a nucleotide sequence consisting of either (a) a fragment of a nucleic acid sequence selected from the group consisting of SEQ ID NOs: 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101; 102, 103, 104, 105, 106, 107, 108, 109, 110, 111, 112, 113, 114, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325 and 326 or (b) the complement of (a); and said nucleic acid 5′-X—Y—Z-3′ is neither (i) a fragment of either a nucleic acid sequence selected from the group consisting of SEQ ID NOs: 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325 and 326 nor (ii) the complement of (i). The —X— and/or —Z— moieties may comprise a promoter sequence (or its complement).

The invention includes nucleic acid comprising sequences complementary to these sequences (e.g. for antisense or probing, or for use as primers).

Nucleic acid according to the invention can take various forms (e.g. single-stranded, double-stranded, vectors, primers, probes, labelled etc.). Nucleic acids of the invention may be circular or branched, but will generally be linear. Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids of the invention are preferably provided in purified or substantially purified form i.e. substantially free from other nucleic acids (e.g. free from naturally-occurring nucleic acids), particularly from other GAS or host cell nucleic acids, generally being at least about 50% pure (by weight), and usually at least about 90% pure. Nucleic acids of the invention are preferably GAS nucleic acids.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acid of the invention may be attached to a solid support (e.g. a bead, plate, filter, film, slide, microarray support, resin, etc.). Nucleic acid of the invention may be labelled e.g. with a radioactive or fluorescent label, or a biotin label. This is particularly useful where the nucleic acid is to be used in detection techniques e.g. where the nucleic acid is a primer or as a probe.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the invention includes mRNA, tRNA, rRNA, ribozymes, DNA, cDNA, recombinant nucleic acids, branched nucleic acids, plasmids, vectors, probes, primers, etc. Where nucleic acid of the invention takes the form of RNA, it may or may not have a 5′ cap.

Nucleic acids of the invention comprise sequences, but they may also comprise non-GAS sequences (e.g. in nucleic acids of formula 5′-X—Y—Z-3′, as defined above). This is particularly useful for primers, which may thus comprise a first sequence complementary to a nucleic acid target and a second sequence which is not complementary to the nucleic acid target. Any such non-complementary sequences in the primer are preferably 5′ to the complementary sequences. Typical non-complementary sequences comprise restriction sites or promoter sequences.

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. Preferred vectors are plasmids. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

Where a nucleic acid is DNA, it will be appreciated that “U” in a RNA sequence will be replaced by “T” in the DNA. Similarly; where a nucleic acid is RNA, it will be appreciated that “T” in a DNA sequence will be replaced by “U” in the RNA.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T).

Nucleic acids of the invention can be used, for example: to produce polypeptides; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides.

The invention provides a process for producing nucleic acid of the invention, wherein the nucleic acid is synthesised in part or in whole using chemical means.

The invention provides vectors comprising nucleotide sequences of the invention (e.g. cloning or expression vectors) and host cells transformed with such vectors.

The invention also provides a kit comprising primers (e.g. PCR primers) for amplifying a template sequence contained within an GAS nucleic acid sequence, the kit comprising a first primer and a second primer, wherein the first primer is substantially complementary to said template sequence and the second primer is substantially complementary to a complement of said template sequence, wherein the parts of said primers which have substantial complementarity define the termini of the template sequence to be amplified. The first primer and/or the second primer may include a detectable label (e.g. a fluorescent label).

For certain embodiments of the invention, nucleic acids are preferably at least 7 nucleotides in length (e.g. 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 90, 100, 110, 120, 130, 140, 150, 160, 170, 180, 190, 200, 225, 250, 275, 300 nucleotides or longer).

For certain embodiments of the invention, nucleic acids are preferably at most 500 nucleotides in length (e.g. 450, 400, 350, 300, 250, 200, 150, 140, 130, 120, 110, 100, 90, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15 nucleotides or shorter).

Primers and probes of the invention, and other nucleic acids used for hybridization, are preferably between 10 and 30 nucleotides in length (e.g. 10, 11, 12, 13, 14, 15, 16, 17, 18, 19, 20, 21, 22, 23, 24, 25, 26, 27, 28, 29, or 30 nucleotides).

### Antisera

Existing T-typing sera are raised against streptococci that have been treated with trypsin. As shown below, and also in ref. 7, these sera recognise both the Pbp and Pap1 proteins. Because we have shown that T-typing is based on the Pbp protein then it is now possible to provide T-typing sera that recognise Pbp but do not recognise Pap1. Thus the invention provides an antibody that (i) binds to only one of SEQ ID NOs: 1 to 13, and (ii) does not bind to Pap1. The lack of binding to the 13 other Pbp sequences permits T-typing by using this antibody. The lack of binding to Pap1 distinguishes the antibody from existing T-typing sera.

Antibodies of the invention may be polyclonal or monoclonal. Monoclonal antibodies are particularly useful in identification and purification of the individual polypeptides against which they are directed. Monoclonal antibodies of the invention may also be employed as reagents in immunoassays, radioimmunoassays (RIA) or enzyme-linked immunosorbent assays (ELISA), etc. In these applications, the antibodies can be labelled with an analytically-detectable reagent such as a radioisotope, a fluorescent molecule or an enzyme. The monoclonal antibodies produced by the above method may also be used for the molecular identification and characterization (epitope mapping) of polypeptides of the invention.

Antibodies of the invention are preferably provided in purified or substantially purified form. Typically, the antibody will be present in a composition that is substantially free of other polypeptides e.g. where less than 90% (by weight), usually less than 60% and more usually less than 50% of the composition is made up of other polypeptides.

The invention also provides a collection (e.g. in the form of a kit) of a plurality of antibodies, wherein each of said plurality (i) binds to only one of SEQ ID NOs: 1 to 13, (ii) does not bind to Pap1. Preferably the plurality includes at least two antibodies, each of which binds to a different one of SEQ ID NOs: 1 to 13. The collection may further comprise antibodies that do not meet criteria (i) and (ii). The collection may be used for T-typing of GAS e.g. by immunoblot, by FACS, etc.

### Multi Pilus Vaccines

It has been shown that immunization with pilus components of the three major streptococcal pathogens (GAS, GBS and S. pneumoniae) can confer type-specific protection. A combination of different Pbp proteins may represent a viable vaccine capable of giving broad coverage against the most important strains involved in disease. 12 Pbp variants account for at least 24 of the 27 most prevalent M-types, and so a combination of these variants could protect against 98% of the circulating strains.

Thus the invention provides a mixture of a plurality of different polypeptides of the invention. A combination of at least two different Pbp variants (e.g. 2, 3, 4, 5, 6, 7, 8, 9, 10, 11, 12 or more) may be used as a vaccine against S. pyogenes disease.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x+10%.

Unless specifically stated, a process comprising a step of mixing two or more components does not require any specific order of mixing. Thus components can be mixed in any order. Where there are three components then two components can be combined with each other, and then the combination may be combined with the third component, etc.

Identity between polypeptide sequences is preferably determined by the Smith-Waterman homology search algorithm as implemented in the MPSRCH program (Oxford Molecular), using an affine gap search with parameters gap open penalty=12 and gap extension penalty=1.

### BRIEF DESCRIPTION OF THE DRAWINGS

FIG. 1 shows the genetic environment of the pbp gene in different FCT-type strains of GAS.

FIG. 2 shows how T-sera recognised recombinant Pbp from various strains in western blot.

FIG. 3 shows the results of a normal T-typing assay for strains classified by their pbp variant type.

FIG. 4 shows alignments of Pbp genes. FIG. 4A aligns FCT-3 and FCT-4 strains. FIG. 4B aligns FCT-6 and FCT-9 strains. FIG. 4C aligns FCT-1 strains.

FIG. 5 shows results of PCR using variant-specific primers.

FIG. 6 shows results of multiplex PCR using a mixture of variant-specific primers.

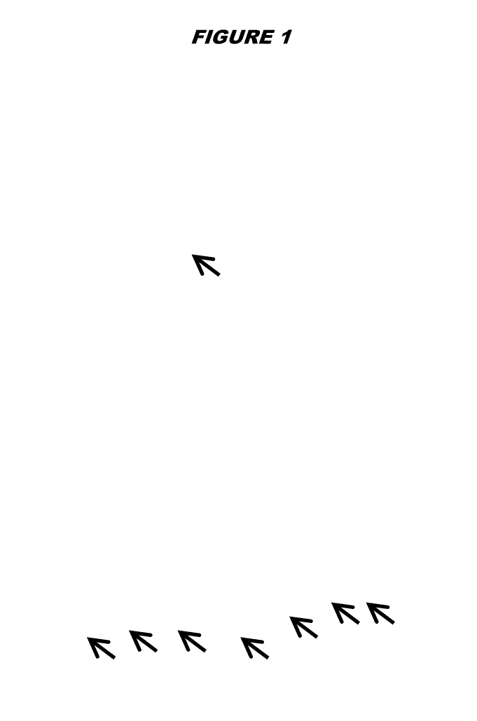

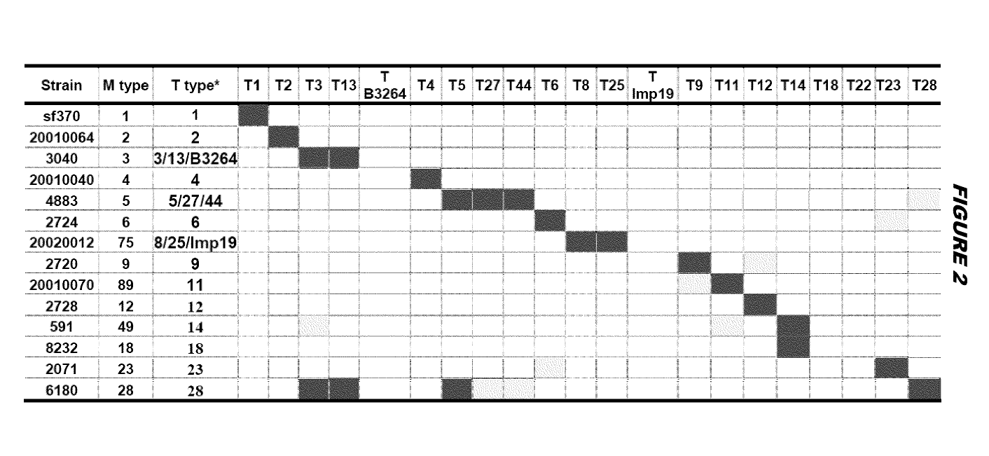

### MODES FOR CARRYING OUT THE INVENTION

### Sequence Variation in the Pilus Backbone Protein (Pbp)

Genes coding for the pilus structural components (pbp, pap1 and pap2) were investigated for 57 different GAS strains with a variety of FCT genotypes and M-serotypes. The primers used for PCR amplification of the pilus regions are shown in Table I. The Pbp genes for the 57 strains are SEQ ID NOs: 58 to 114, encoding amino acid sequences SEQ ID NOs: 1 to 57 respectively.

These 57 sequences were found to group into 15 variants. Prototypic sequences for each variant are SEQ ID NOs: 1 to 15. Table II herein shows the amino acid identity derived from pairwise comparisons between these 15 variants. Within a single M-type the Pbp protein varied by no more than 2/100 amino acids between strains, although the DNA sequence revealed several silent single nucleotide differences. In four cases the same variant was found in different M-types, again with greater than 98% identity.

Pbp from a M18-type strain (SEQ ID NO: 11) had 90% identity to Pbp from a M49-type strain (SEQ ID NO: 14). Rather than classify these sequences as different variants, immunological cross-reactivity between the strains led to their being classified as a single variant.

In contrast to the high level of intra-variant amino acid sequence identity, between different Pbp variants there is much less conservation, reaching a maximum of 72% in tested sequences.

The sequences of the Pap1 protein (SEQ ID NOs 179-216, encoded by SEQ ID NOs 253-290) did not divide as clearly into distinct variants and a wider spread of pairwise identities were observed.

The sequences of Pap2 protein (SEQ ID NOs 217-252, encoded by SEQ ID NOs 291-326) did not correlate with T-types.

Different Backbone Proteins are Associated with Different T-Serotypes.

From the analysis of M-types associated with each Pbp variant, we hypothesized that M-types which share the same backbone might also share the same T-serotype, independent of the FCT type carried and ancillary protein sequences.

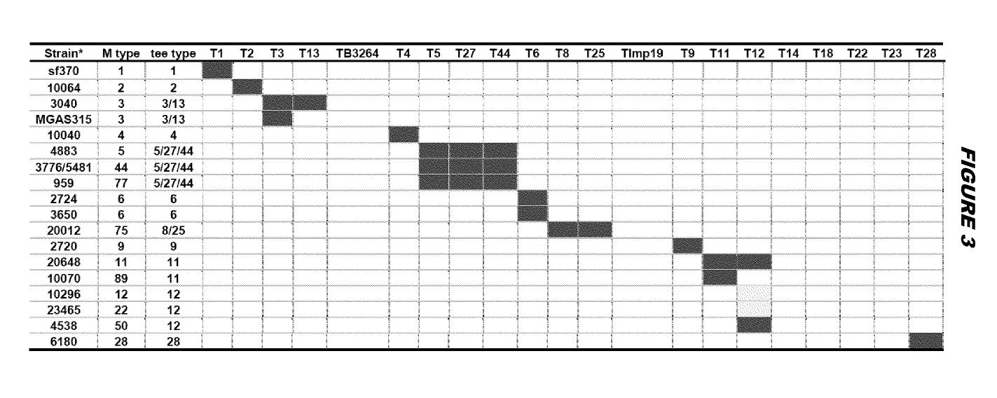

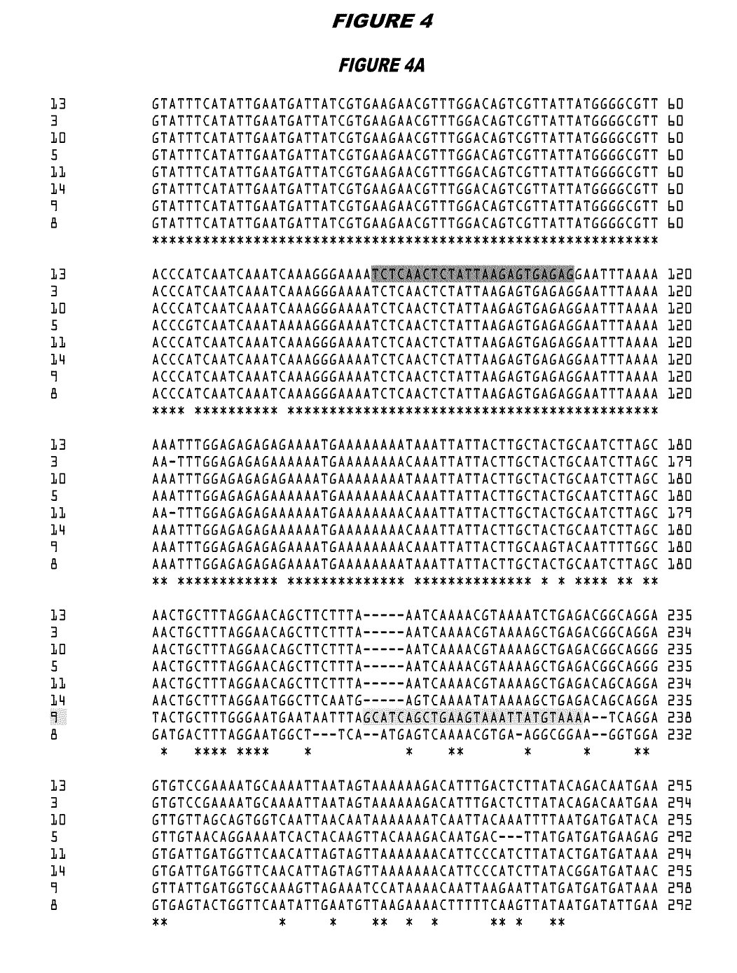

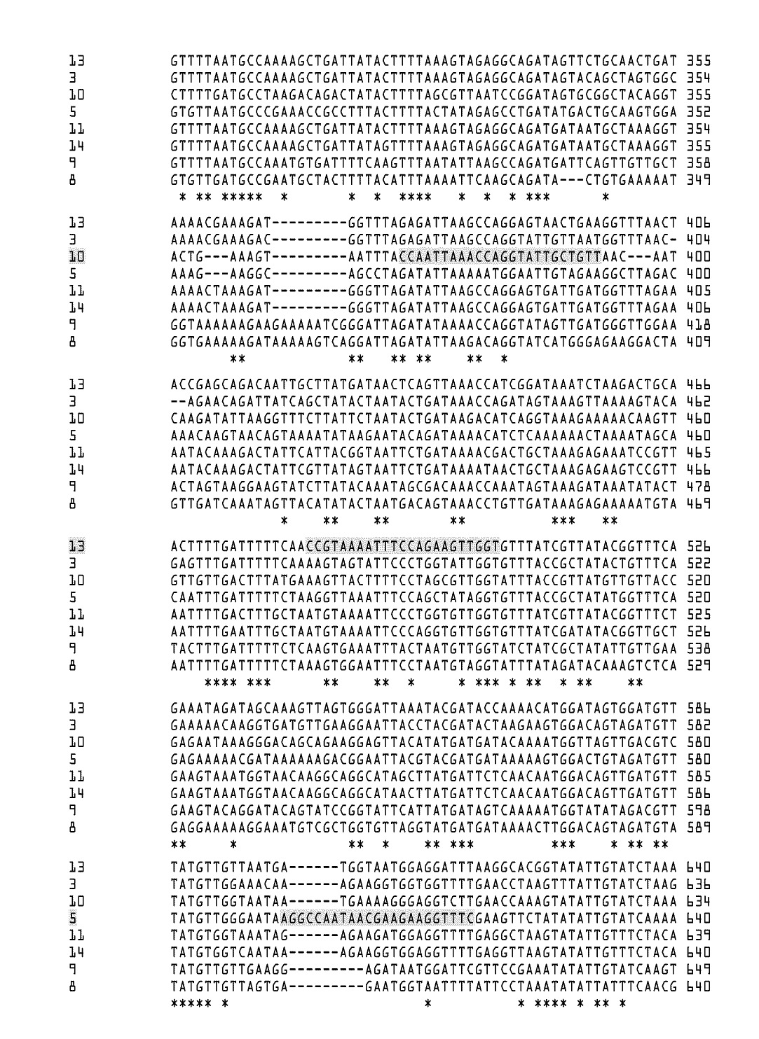

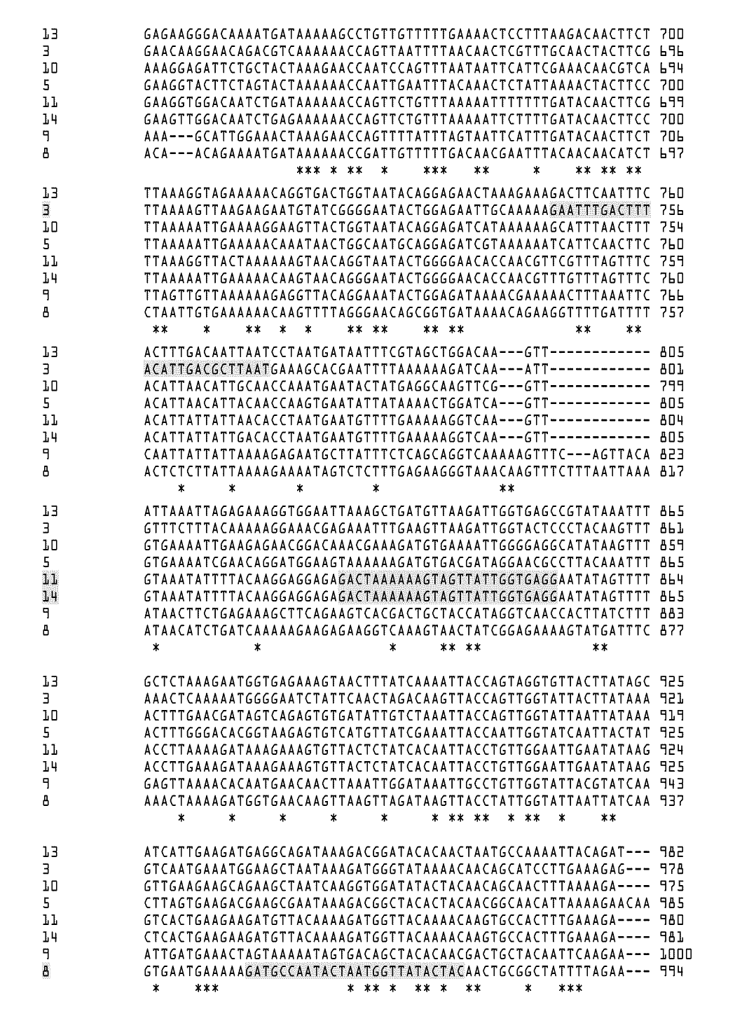

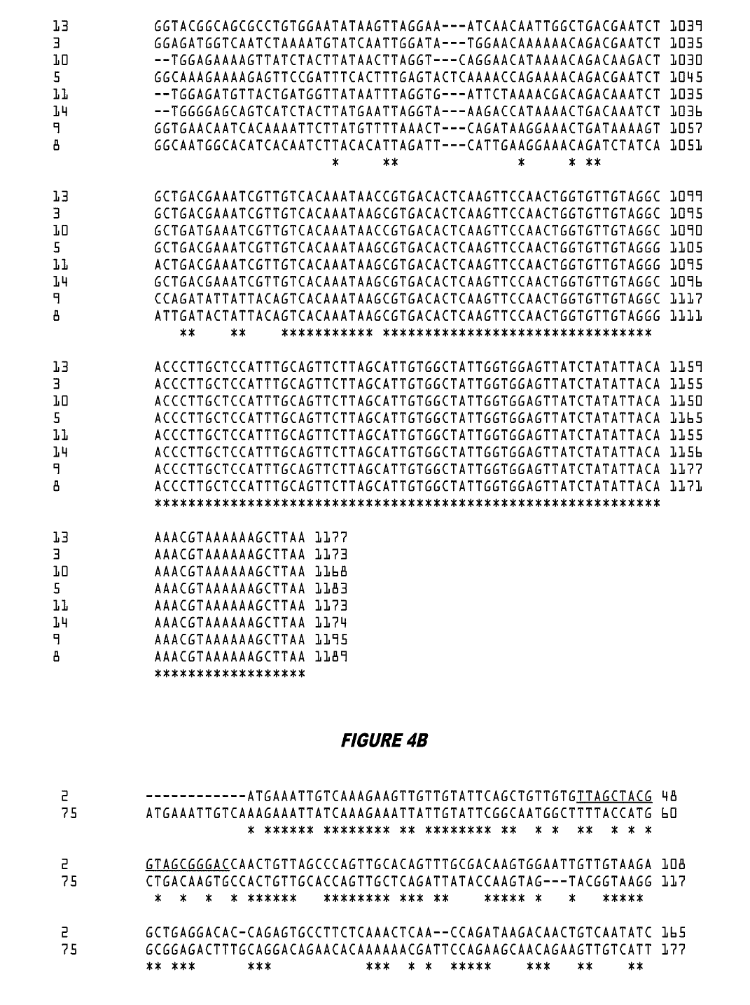

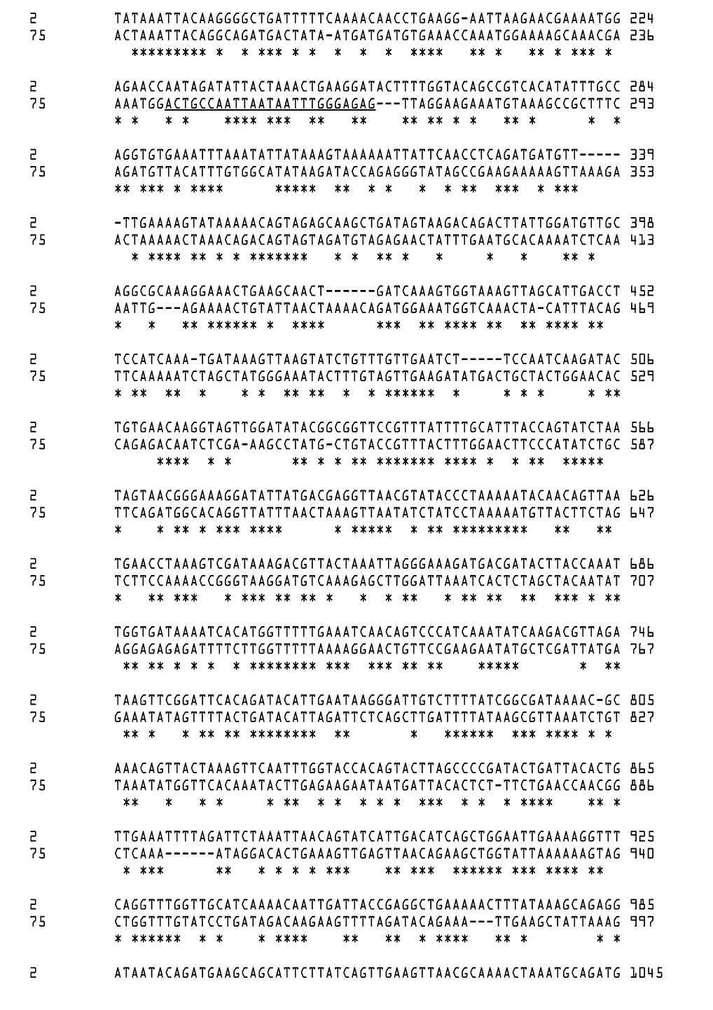

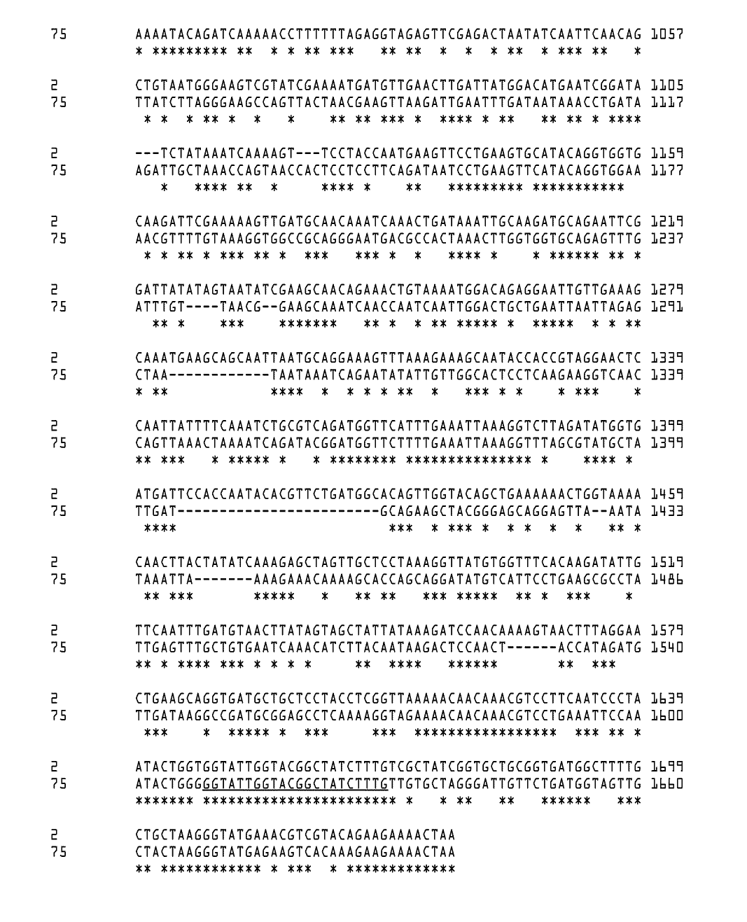

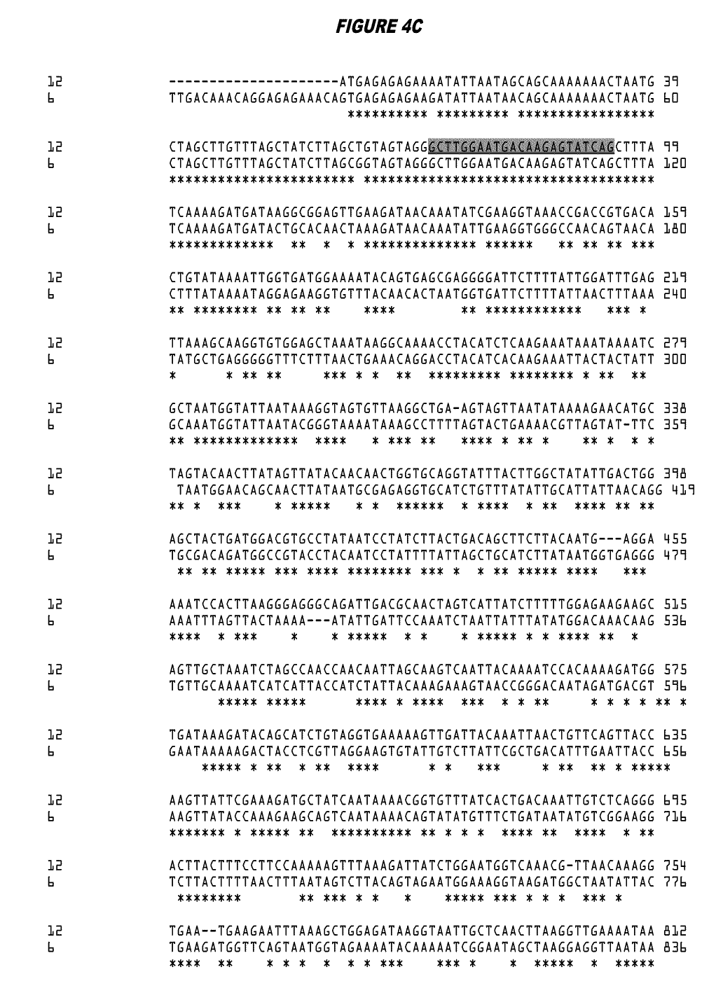

14 pbp genes (SEQ ID NOs: 1 to 14) were cloned and expressed in E. coli and the resulting purified recombinant proteins were tested in immunoblot with each of the 21 commercially available T-typing antisera. The results are summarised in FIG. 2. In most cases each Pbp was recognized by a single T-typing serum, except that the Pbp that reacted with serum T-28 also reacted non-specifically with some other T antisera. The Pbp sequences from M-49 and M-18 types, which have an amino acid identity of 90%, were recognized by the same T serum hence justifying considering these two proteins as a single Pbp variant. In three cases, a single Pbp reacted with more than one T-serum. However, in each case the positive sera belonged to the known families of related T-sera which are usually grouped together (T3/13/B3264, T5/27/44, T8/25/Imp19).

Thus there is a clear correlation between the T-serotype and specific Pbp variants sharing homology of at least 90%.

T-antisera also recognised the Pap1 ancillary protein 1, but did not recognise Pap2. Unlike Pbp, though, there was no close correlation between T-type and Pap1 variants.

T-Serotype Agglutination Specificity Correlates with Pbp

Because of the correlation between T-serotype and Pbp variants in the western blot experiments, the standard agglutination reaction (on which T-typing is based) was performed using various GAS strains which had already been classified according to their Pbp variant. As shown in FIG. 3, a strict correspondence between a Pbp variant and T-agglutination was observed. Thus the sequence of the pbp gene is sufficient to predict the T-type.

To test this idea, we looked at a strain (M50_{—}4538) that had not been tested in the western blot experiments. Its pbp gene was sequenced and its T-type was predicted. Agglutination confirmed that the prediction was correct.

T-typing sera 5, 27 and 44 are known to cross-react. These three sera reacted with three strains that, while having different M-types and different pap1 sequences, shared an identical pbp sequence. So far we did not find a pbp protein that reacts with T-typing sera 18 or 22, but the latter occurs in only 1/4000 strains [31].

Thus, although T-typing sera recognise both the pap1 and the pbp genes, it is the pbp product that determines the T-serotype.

### PCR Detection of Tee-Types

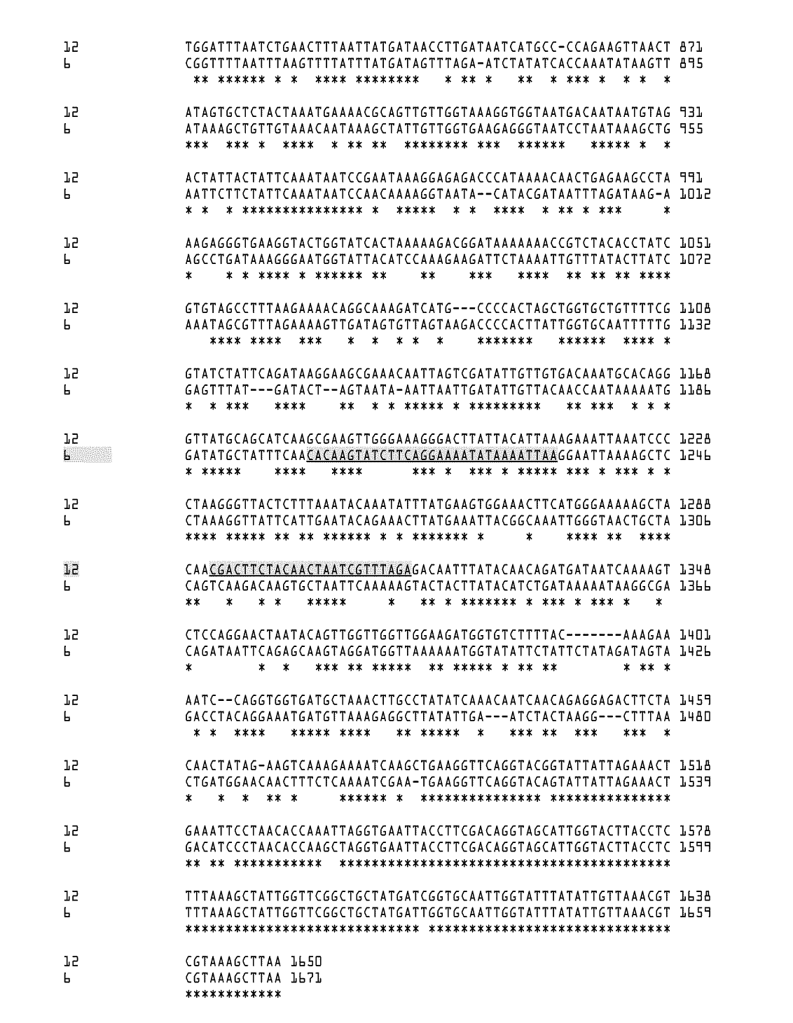

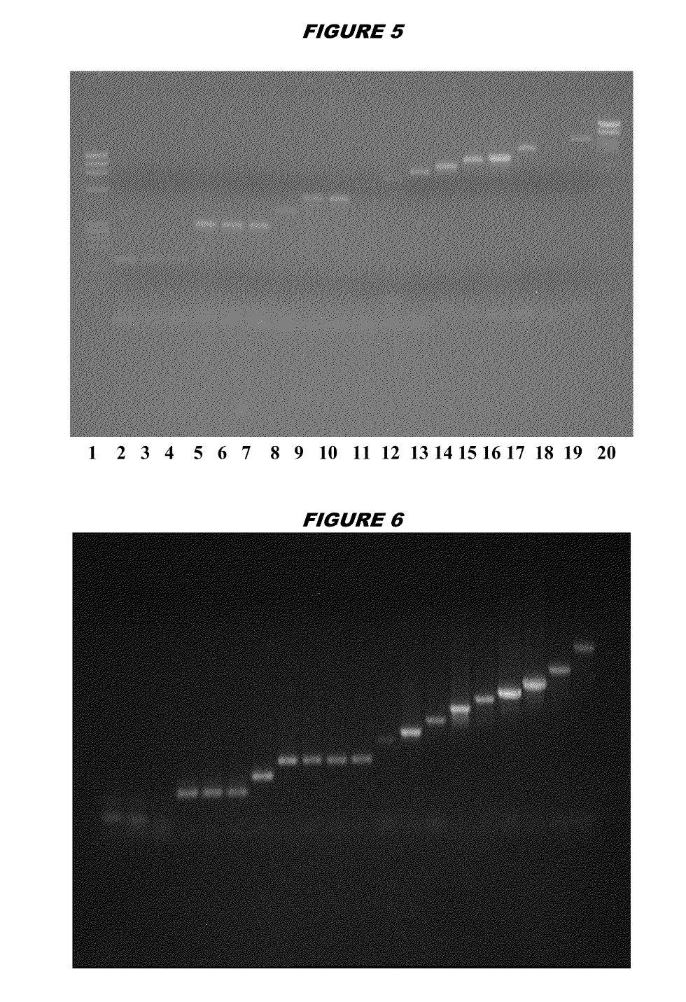

Bacterial DNA from the following strains was incubated with the relevant PCR primer pairs described above and selected from SEQ ID NOs: 161 to 178. Results of amplification are shown in FIG. 5. Lanes 1 and 20 include molecular weight markers and lane 18 is empty. The other lanes show amplification from strains: (2) 2727; (3) 3789; (4) 20010070; (5) 2728; (6) 20023465; (7) 4538; (8) 6180; (9) 3776/27/44; (10) 5481/27/44; (11) 2724/13; (12) 8232; (13) 2720; (14) SF370; (15) 3040; (16) DS71; (17) 20010012/25; (19) 20010040. Amplification was successful with these primers for these strains.

A multiplex experiment was also performed, in which a mixture of all primers was used. The results are shown in FIG. 6. Each lane shows results obtained with a single strain. The lanes are ordered by amplicon sizes, which are as follows: 145 bp=M11(2727), M78(3789), M89(20010070); 308 bp=M12(2728), M22(20023465), M50(4538); 395 bp=M28(4436); 530 bp=M5(4883), M44(3776), M44(5481), M77(4959); 685 bp=M3(3040); 767 bp=M18(8232); 865 bp=M9(2720); 1024 bp=M1(SF370); 1144 bp=M6(2724); 1245 bp=M23(DSM2071); 1338 bp=M75(20010012); 1630 bp=M2(20010064); 2151 bp=M4(20010040). FIG. 6 confirms that the primers can amplify their target strains even in the presence of primers for other targets.

It will be understood that the invention has been described by way of example only and modifications may be made whilst remaining within the scope and spirit of the invention.

### REFERENCES

[1] Lancefield, R. C., The antigenic complex of Streptococcus hemolyticus. I. Demonstration of a type-specific substance in extracts of Streptococcus hemolyticus. J Exp Med, 1928. 47: p. 9-10.
[2] Lancefield, R. C. and V. P. Dole, The properties of T antigen extracted from group A hemolytic streptococci. J Exp Med, 1946. 84: p. 449-471.
[3] Griffith, F., The serological classification of streptococcus pyogenes. J. Hyg., 1934. 34:: p. 542-584.
[4] Stevens, D. L., M. H. Tanner, J. Winship, R. Swans, K. M. Ries, and a.E.L.K. P. M. Schlivert, Severe group A streptococcal infections associated with a toxic shock-like syndrome and scarlet fever toxin A. N. Engl. J. Med., 1989. 321: p. 1-7.
[5] Neal, S., et al., International Quality Assurance Study for Characterization of Streptococcus pyogenes. J Clin Microbiol, 2007. 45(4): p. 1175-9.
[6] Johnson, D. R., et al., Characterization of group A streptococci (Streptococcus pyogenes): correlation of M-protein and emm-gene type with T-protein agglutination pattern and serum opacity factor. J Med Microbiol, 2006. 55(Pt 2): p. 157-64.
[7] Lizano et al. (2007) J Bacteriol, 189(4):1426-34.
[8] Schneewind, O., K. F. Jones, and V. A. Fischetti, Sequence and structural characteristics of the trypsin-resistant T6 surface protein of group A streptococci. J Bacteriol, 1990. 172(6): p. 3310-7.
[9] Mora, M., et al., Group A Streptococcus produce pilus-like structures containing protective antigens and Lancefield T antigens. Proc Natl Acad Sci USA, 2005. 102(43): p. 15641-6.
[10] Beall et al. (1996) J Clin Microbiol 34:953-8.
[11] Geysen et al. (1984) PNAS USA 81:3998-4002.
[12] Carter (1994) Methods Mol Biol 36:207-223.
[13] Jameson, B A et al. 1988, CABIOS 4(1):181-186.
[14] Raddrizzani & Hammer (2000) Brief Bioinform 1(2):179-189.
[15] De Lalla et al., (1999) J. Immunol. 163:1725-1729.
[16] Brusic et al (1998) Bioinformatics 14(2):121-130
[17] Meister et al. (1995) Vaccine 13(6):581-591.
[18] Roberts et al. (1996) AIDS Res Hum Retroviruses 12(7):593-610.
[19] Maksyutov & Zagrebelnaya (1993) Comput Appl Biosci 9(3):291-297.
[20] Feller & de la Cruz (1991) Nature 349(6311):720-721.
[21] Hopp (1993) Peptide Research 6:183-190.
[22] Welling et al. (1985) FEBS Lett. 188:215-218.
[23] Davenport et al. (1995) Immunogenetics 42:392-397.
[24] Bodanszky (1993) Principles of Peptide Synthesis (ISBN: 0387564314).
[25] Fields et al. (1997) Meth Enzymol 289: Solid-Phase Peptide Synthesis. ISBN: 0121821900.
[26] Chan & White (2000) Fmoc Solid Phase Peptide Synthesis. ISBN: 0199637245.
[27] Kullmann (1987) Enzymatic Peptide Synthesis. ISBN: 0849368413.
[28] Ibba (1996) Biotechnol Genet Eng Rev 13:197-216.
[29] U.S. Pat. No. 5,707,829
[30] Current Protocols in Molecular Biology (F. M. Ausubel et al. eds., 1987) Supplement 30.
[31] Johnson et al. (2006) J Med Microbiol, 55:157-64.

TABLE I

| Strain | Primer | Primer Sequence | SEQ ID |
|---|---|---|---|
| SF37C | Pbp_for | GTGCGTCATATGGCTACAACAGTTCACGG | 115 |
| | Pbp_rev | GCGTCTCGAGAAAGTCTTTTTTATTTGTAAAAGTAATG | 116 |
| | Pap1_for | GTGCGTCATATGGCTAAGACTGTTTTTGGT | 117 |
| | Pap1_rev | GCGTCTCGAGGCCATTGATCTTTTGA | 118 |
| 20010064 | Pbp_for | GTGCGTCATATGGAGGACACCAGAGTGCCT | 119 |
| | Pbp_rev | GCGTCTCGAGTGAAGGACGTTTGTTGTTTTTAACCGA | 120 |
| | Pap1_for | GTGCGTGCTAGCGAAACTCAAGATAACAATCCAGCAC | 121 |
| | Pap1_rev | GCGTCTCGAGAACACTCGGTGGACGTTTTG | 122 |
| 3040 | Pbp_for | GTGCGTCATATGGAGACGGCAGGAGTG | 123 |
| | Pbp_rev | GCGTCTCGAGAGCTTTTTTACGTTTTGTAATATAG | 124 |
| | Pap1_for | GTGCGTGCTAGCGCTGAAGAACAATCAGTG | 125 |
| | Pap1_rev | GCGTCTCGAGAAGATCTTTTCGGTTTTC | 126 |
| 20010040 | Pbp_for | GTGCGTCATATGGAAGCAGAATCATCACATAAAACCGA | 127 |
| | Pbp_rev | GCGTCTCGAGGGTGATTTTTTTGTTAATCACCCGTTG | 128 |
| | Pap1_for | GTGCGTCATATGTTATCAGGACATTCGAGGTCA | 129 |
| | Pap1_rev | GCGTCTCGAGAACAAACTTTTTATTAATAATCTCATTAAATTG | 130 |
| 4883 | Pbp_for | GTGCGTCATATGGAGACGGCAGGGGT | 131 |
| | Pbp_rev | GCGTCTCGAGAGTGTCACGCTTATTTGT | 132 |
| 2724 | Pbp_for | GTGCGTCATATGAAAGATGATACTGCACAACT | 133 |
| | Pbp_rev | GCGTCTCGAGTTCACCTAGCTTGGTGTTAG | 134 |
| | Pap1_for | GTGCGTCATATGTACAGTAGATTGAAGAGAGAGTTAG | 135 |
| | Pap1_rev | GCGTCTCGAGCTGATAAATCTTATAATTTTTAATCATG | 136 |
| 2720 | Ppb_for | GTGCGTCATATGGAGTACTGGTTCAATATTGAATGTTAAG | 137 |
| | Pbp_rev | GCGTCTCGAGAGTGTCACGCTTATTTGTGACTG | 138 |
| | Pap1_for | GTGCGTGCTAGCCAAAGCATATTTGGAGAGGAAAAGAG | 139 |
| | Pap1_rev | ACTCGCTAGCGGCCGCAAGATCTTTTCGGTTTTCAAAAGCTAC | 140 |
| 2728 | Ppb_for | GTGCGTGCTAGCGAGACGGCAGGGGT | 141 |
| | Pbp_rev | GCGTCTCGAGAGTGTCACGGTTATTTG | 142 |
| | Pap1_for | GTGCGTGCTAGCCAAAGCATATTTGGAGAG | 143 |
| | Pap1_rev | GCGTCTCGAGAAGATCTTTACGGTTTTCA | 144 |
| Dsm2071 | Pbp_for | GTGCGTCATATGTTATCAAAAGATGATAAGGCGGAG | 145 |
| | Pbp_rev | GCGTCTCGAGTTCACCTAATTTGGTGTTAGGAATTTC | 146 |
| 6180 | Pbp_for | GTGCGTCATATGACAGCTTCTTTAAATCAAAACGTAAAATCTGAG | 147 |
| | Pbp_rev | GCGTCTCGAGTTGAGTGTCACGGTTATTTGTGAC | 148 |
| | Pap1_for | GTGCGTCATATGGCTCACGAATTGGTTGAGGT | 149 |
| | Pap1_rev | GCGTCTCGAGAAGATCTTTACGGTTTTCAAAAGTGAC | 150 |
| 4538 | Pap1_for | GTGCGTGCTAGCGCTCACGAATTGGTTGAGGTAC | 151 |
| | Pap1_rev | GCGTCTCGAGAAGATCTTTTCGGTTTTCAAAAGTGAC | 152 |
| 20010012 | Pbp_for | GTGCGTCATATGGAGACTTTGCAGGACAGAAC | 153 |
| | Pbp_rev | GCGTCTCGAGTTCAGGACGTTTGTTGTTTTCTAC | 154 |
| 4959 | Ppb_for | GTGCGTCATATGGAGACGGCAGGGGT | 155 |
| | Ppb_rev | GCGTCTCGAGAGTGTCACGCTTATTTGT | 156 |
| | Pap1_for | GTGCGTCATATGGCTGAAGAAAAATCTACTG | 157 |
| | Pap1_rev | GCGTCTCGAGAAGATCTTTTCGGTTTTC | 158 |
| 20010070 | Pbp_for | GTGCGTCATATGGAAGTAAATTATGTAAAATCAGGAGTTATTG | 159 |
| | Pbp_rev | GCGTCTCGAGAGTGTCACGCTTATTTGTGACTG | 160 |

TABLE II

| | | | FCT3 | | | | | | | | | | | | | |
|---|---|---|---|---|---|---|---|---|---|---|---|---|---|---|---|---|
| | | FCT2 | | | M18, | M33, | | FCT4 | | | | FCT6 | FCT5 | FCT1 | | FCT9 |
| | ID % | M1 | M3 | M5, 44, 77 | 49 | 53 | stD33 | M9 | M11, 78, 89 | M12, 22, 50 | M28 | M2 | M4 | M6 | M23 | M75 |
| FCT2 | M1 | 100 | 39 | 39 | 36 | 39 | 38 | 39 | 38 | 40 | 40 | 21 | 23 | 23 | 20 | 24 |
| FCT3 | M3 | | >99 | 64 | 67 | 68 | 67 | 60 | 55 | 61 | 72 | 20 | 24 | 25 | 25 | 27 |
| | M5, 44, 77 | | | >99 | 61 | 69 | 64 | 55 | 50 | 65 | 62 | 25 | 23 | 23 | 25 | 23 |
| | M18, 49 | | | | >90 | 65 | 65 | 58 | 54 | 62 | 65 | 24 | 23 | 24 | 23 | 25 |
| | M33, 53 | | | | | >97 | 63 | 55 | 52 | 64 | 63 | 26 | 23 | 26 | 23 | 30 |
| | stD33 | | | | | | 100 | 56 | 55 | 62 | 81 | 26 | 22 | 24 | 24 | 22 |
| FCT4 | M9 | | | | | | | 100 | 57 | 53 | 56 | 22 | 24 | 27 | 23 | 23 |
| | M11, 78, 89 | | | | | | | | >99 | 51 | 52 | 23 | 20 | 24 | 21 | 26 |
| | M12, 22, 50 | | | | | | | | | >97 | 58 | 26 | 21 | 26 | 23 | 25 |
| | M28 | | | | | | | | | | >99 | 23 | 24 | 23 | 23 | 22 |
| FCT6 | M2 | | | | | | | | | | | 100 | 26 | 21 | 24 | 40 |
| FCT5 | M4 | | | | | | | | | | | | 100 | 24 | 25 | 24 |
| FCT1 | M6 | | | | | | | | | | | | | >99 | 55 | 23 |
| | M23 | | | | | | | | | | | | | | 100 | 24 |
| FCT9 | M75 | | | | | | | | | | | | | | | 100 |

TABLE III
SEQUENCE LISTING AND STRAINS FOR PBP

| SEQ ID | Strain |
|---|---|
| 1 | M1-SF370 |
| 2 | M2-10270 |
| 3 | M3-315 |
| 4 | M4-10750 |
| 5 | M5-Manfredo |
| 6 | M6-10394 |
| 7 | M75-20010012 |
| 8 | M9-2720 |
| 9 | M11-2727 |
| 10 | M12-A735 |
| 11 | M18-8232 |
| 12 | M23-DSM2071 |
| 13 | M28-6180 |
| 14 | M49-591 |
| 15 | M33-29487 |
| 16 | M1-5005 |
| 17 | M12-2096 |
| 18 | M12-9429 |
| 19 | M12-2728* |
| 20 | M12-20010296* |
| 21 | M12-20020069* |
| 22 | M2-20010064 |
| 23 | M2-20010065 |
| 24 | M2-20010194 |
| 25 | M2-20030561 |
| 26 | M22-20020641 |
| 27 | M22-20023465 |
| 28 | M22-20023621 |
| 29 | M28-20010164 |
| 30 | M28-20010218 |
| 31 | M28-20030176 |
| 32 | M28-20030574 |
| 33 | M28-20030902 |
| 34 | M28-4436 |
| 35 | M3-2721 |
| 36 | M3-3040 |
| 37 | M3-SSI |
| 38 | M4-20010040 |
| 39 | M4-20010092 |
| 40 | M4-20030968 |
| 41 | M44-3776 |
| 42 | M44-5481 |
| 43 | M5-4883 |
| 44 | M50-4538 |
| 45 | M53-ALAB49 |
| 46 | M6-2724 |
| 47 | M6-3650 |
| 48 | M6-D471 |
| 49 | M77-4959 |
| 50 | M78-3789 |
| 51 | M89-20010070 |
| 52 | M89-20021915 |
| 53 | M89-20023717 |
| 54 | M89-20030266 |
| 55 | M89-20030382 |
| 56 | STD633-D633 |
| 57 | M1-3348 |

*Partial

1. A method for determining the T-classification of a Streptococcus pyogenes bacterium, wherein the sequence of the bacterium's pbp gene is determined in whole or in part.

2. A method for analysing a Streptococcus pyogenes bacterium, comprising a step in which the sequence of the bacterium's pbp gene is determined in whole or in part.

3. A method for determining the T-classification of a Streptococcus pyogenes bacterium, wherein the sequence, in whole or in part, of the bacterium's pbp gene is compared to one or more known pbp sequence(s) that have been correlated with T serotypes.

4. A kit for analysing a Streptococcus pyogenes bacterium, comprising primers for amplifying a nucleic acid sequence comprising the whole of part of a pbp gene from a Streptococcus pyogenes bacterium.

5. A method for determining if a Streptococcus pyogenes bacterium has a particular T serotype, comprising a step in which the sequence of the bacterium's pbp gene is compared to the sequence of a pbp gene from a Streptococcus pyogenes strain that is known to have the particular T serotype, with a sequence match indicating that the bacterium has the particular T serotype and no sequence match indicating that the bacterium does not have the particular T serotype.

6. A method for determining if a Streptococcus pyogenes bacterium has a particular T serotype, comprising a step in which the bacterium's pbp gene is contacted with a nucleic acid that, under the conditions of this step, gives a first signal when contacted with one of SEQ ID NOs: 58 to 70 and a second signal when contacted with each of the other SEQ ID NOs: 58 to 70, wherein the first and second signals can be distinguished from each other.

7. (canceled)

8. The method of claim 6 wherein the T serotype is T serotype 2.

9. The method of claim 6 wherein the T serotype is T serotype 3/13.

10. The method of claim 6 wherein the T serotype is T serotype 4.

11. The method of claim 6 wherein the T serotype is T serotype 5/27/44.

12. The method of claim 6 wherein the T serotype is T serotype 6.

13. The method of claim 6 wherein the T serotype is T serotype 8 or 25.

14. The method of claim 6 wherein the T serotype is T serotype 9.

15. The method of claim 6 wherein the T serotype is T serotype 11.

16. The method of claim 6 wherein the T serotype is T serotype 12.

17. The method of claim 6 wherein the T serotype is T serotype 14.

18. The method of claim 6 wherein the T serotype is T serotype 23.

19. The method of claim 6 wherein the T serotype is T serotype 3/5/13/28.

20. A kit comprising primers for amplifying a template sequence comprising at least a part of the S. pyogenes pbp gene, the kit comprising a first primer and a second primer, wherein the first primer comprises a sequence substantially complementary to a portion of said template sequence and the second primer comprises a sequence substantially complementary to a portion of the complement of said template sequence, wherein the sequences within said primers which have substantial complementarity define the termini of the template sequence to be amplified.

21. An isolated polypeptide selected from the group consisting of:
a polypeptide comprising an amino acid sequence having at least 50% sequence identity to an amino acid sequence selected from the group consisting of SEQ ID NOs: 1, 2, 4, 6, 7, 8, 9, 10, 11, 12, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 and 57; and
a polypeptide comprising a fragment of at least 7 consecutive amino acids of an amino acid sequence selected from the group consisting of SEQ ID NOs: 1, 2, 4, 6, 7, 8, 9, 10, 11, 12, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 and 57.

22. (canceled)

23. An isolated nucleic acid comprising a nucleotide sequence encoding the polypeptides of claim 21.

24. An isolated antibody that (i) binds to only one of SEQ ID NOs: 1 to 13, and (ii) does not bind to Pap 1.

25. A mixture of a plurality of different isolated polypeptides according to claim 21.

26. The method of claim 7 wherein the T serotype is T serotype 1.
